(19) United States
(12) Patent Application Publication
(10) Pub. No.: US 2002/0048773 A1
(43) Pub. Date: Apr. 25, 2002

(54) Method and apparatus for facile manipulation of spatially addressed solid supports for combinatorial chemical synthesis

(76) Inventor: David Barnes-Seeman, Cambridge, MA (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 09/821,192

(22) Filed: Mar. 29, 2001

Related U.S. Application Data
(60) Provisional application No. 60/242,793, filed on Oct. 24, 2000.

(51) Int. Cl. G01N 33/53; B01J 8/04

(52) U.S. Cl. 435/7.1; 422/189

(57) ABSTRACT

An apparatus and method by which solid supports may be retained in place in an array within an reaction vessel but which allows easy lateral manipulation of the solid supports in a defined manner to new arrays for subsequent steps in the synthesis of combinatorial chemistry libraries of compounds.

### FIELD OF THE INVENTION

The present invention relates to an apparatus and method useful in the field of combinatorial chemistry. More particularly, the present invention relates to methods of handling solid supports during the synthesis of combinatorial chemical libraries of compounds whereby the synthesis is carried out on a plurality of solid supports which in turn are distributed in the form of a series of arrays. The position of each solid support in each array determines the exact identity of each compound.

### BACKGROUND OF THE INVENTION

The screening of chemical libraries to identify compounds which have novel pharmacological and material science properties is a common practice. Those practiced in the art of combinatorial chemistry can accomplish the synthesis of combinatorial chemical libraries using two general methods. These methods are known to those skilled in the art as parallel methods and “split-pool” methods. For reviews of such methods, see Tan et al., Ligand Discovery Using Encoded Combinatorial Libraries, CURRENT OPINION IN DRUG DISCOVERY AND DEVELOPMENT (2000), 3(4), 439-53; Barnes et al., Recent Developments in the Encoding and Deconvolution of Combinatorial Libraries, CURRENT OPINION IN CHEMICAL BIOLOGY, (2000), 4, 346-50; Czarnik et al., Encoding Strategies in Combinatorial Synthesis, PROCEEDINGS OF THE NATIONAL ACADEMY OF SCIENCES (1997), 1, 60-66.

A common feature to the combinatorial library methods is that a unique solid support combination of monomers is reacted to form a single oligomer or compound or, alternately, set of oligomers or compounds at a predefined unique physical location or address in the synthesis process. An example of the parallel method is provided by Geysen et al., “Use of Peptide Synthesis to Probe Viral Antigen for Epitopes to a Resolution of a Single Amino Acid” PROC. NATL. ACAD. SCI. USA 1984, 81, 3998, and involves the generation of peptide libraries on an array of immobilized polymeric pins (a solid support) that fit the dimensions of a 96-well microtiter plate. A two-dimensional matrix of combinations is generated in each microtiter plate experiment, where n×m unique oligomers or compounds are produced for a solid support combination of n+m parallel monomer addition steps. The structure of each of the individual library members is determined by analyzing the pin location and the monomers employed at that address during the sequence of reaction steps in the synthesis. An advantage of this method is that individual oligomer or compound products can be released from the polymeric pin surface in a spatially-addressable manner to allow isolation and screening of each discrete member of the library. Another advantage of this method is that the number of solid supports required is equal to, i.e. no larger than, the number of library members to be synthesized. Thus, relatively large quantities, i.e. micromolar quantities, of individual library members are synthesized in a practical manner using this method. A disadvantage of this method is that the number of wells required is the same as the number of compounds, which makes liquid handling prohibitively expensive for more than tens of thousands of compounds/wells.

Related to the Geysen pin method are the parallel synthesis methods which use a reaction vessel system such as that practiced by Cody, et al., Multiple and Combinatorial Peptide Synthesis. Chemical Development and Biological Applications, METHODS MOL. BIOL. (1994), 36 (Peptide Analysis Protocols), 305-28. The parallel synthesis methods encompass the practice of distributing a quantity of solid support, such as chemically-derivatized polymeric resin beads (namely those of the composition polystyrene, polystyrene grafted with polyethylene glycol, or polyacrylimide, etc.) in a two dimensional matrix of n×m individual reaction vessels allowing the parallel addition of a set of n×m in reactive monomers to produce a set of n x m oligomers or compounds. This parallel method has advantages similar to that of Geysen, et al. Individual oligomer or compound products can therefore be released from the solid support in a spatially-addressable manner to allow isolation and screening of each discrete member of the library. Additionally, the number of solid supports required is equal to, i.e. no larger than, the number of library members to be synthesized. Thus, relatively large quantities, i.e. micromolar to millimolar quantities, of individual library members also are synthesized in a practical manner using this method. As with the Geysen pin method, a disadvantage of this method is that the number of wells required is the same as the number of compounds, which makes liquid handling prohibitively expensive for more than tens of thousands of compounds/wells.

Another example of a spatially-addressable method is the photolithographic method for synthesizing a collection oligomers or compounds on the chemically-derivatized surface of a chip (a solid support) provided by Fodor et al., Light-Directed, Spatially Addressable Parallel Chemical Synthesis, SCIENCE (Feb. 15, 1991), 251(4995), 767-73. A variety of masking strategies can be employed to selectively remove photochemically-labile protecting groups thus revealing reactive functional groups at defined spatial locations on the chip. The functional groups are reacted with a monomer by exposing the chip surface to appropriate reagents. The sequential masking and reaction steps are recorded, thus producing a pre-defined record of discrete oligomers or compounds at known spatial addresses in an experiment. An advantage of this method is that binary masking strategies can be employed to produce 2″ unique oligomers or compounds for n masking and monomer addition cycles. Two important disadvantages of this method are that a) relatively minute quantities are produced on the surface of the chip and; b) release and isolation of individual library members is not technically feasible.

Split-pool combinatorial library methods differ from parallel methods in that the physical location of each unique oligomer or compound is not discrete. Instead, pools of library members are manipulated throughout the experiment. There are two major categories of split-pool methods currently in practice. These are: 1) deconvolution methods pioneered by Furka et. al., The Portioning-Mixing (Split) Method, BIOFORUM INT. (1998), 2(4), 169-172; and Houghten, et. al., Drug Discovery and Vaccine Development Using Mixture-Based Synthetic Combinatorial Libraries, DRUG DISCOVERY TODAY (2000), 5(7) 276-85; and 2) encoded methods by Ni et al., Versatile Approach to Encoding Combinatorial Organic Synthesis Using Chemically Robust Secondary Amine Tags, JOURNAL OF MEDICINAL CHEMISTRY (1996), 39, 1601-8), Nestler et al., A General Method for Molecular Tagging of Encoded Combinatorial Chemistry Libraries, JOURNAL OF ORGANIC CHEMISTRY, (1994), 59, 4723) amongst others.

It is common in the practice to employ solid support-based chemistry for these methods. A collection of solid supports is split into individual pools. These pools are then exposed to a series of reactive monomers, followed by a recombination step, in which the position of all solid supports is randomized. The solid supports are then split into a new set of individual pools, exposed to a new series of reactive monomers, followed by a second recombination step. By repeating this split, react and combine process, all possible combinations of oligomers or compounds from the series of monomers employed are obtained, providing a large excess of solid supports are utilized. The number of oligomers or compounds obtained in an experiment is equal to the product of the monomers employed, however, the number of chemical transformation steps required is only equal to the sum of the monomers employed. Therefore, a geometric amplification of oligomers or compounds is realized relative to the amount of chemical transformation steps employed. For instance, only nine (9) transformation steps were employed using three (3) amino acid monomers in a three step process for the combinatorial synthesis of 27 peptide oligomers.

The prior art non-encoded split-pool methods produce pools of oligomers or compounds as a product of the experiment. Therefore, the identification of a specific member of the library is typically found by screening the pools for a desired activity, biological or otherwise. The disadvantages of the deconvolution split-pool methods are that (a) the technique always requires that large mixtures of oligomers or compounds are screened in bioassays, (b) sequential rounds of resynthesis and bioassay are always required to deconvolute a library, (c) since single oligomers or compounds are not produced a library is always stored as a mixture, requiring later deconvolution, (d) screening mixtures of compounds precludes screening for the specificity with which a compound interacts with a protein or target of interest, and (e) when screening mixtures of compounds for biological activity, a pool containing one very active compound among inactive compounds, may be overlooked because the inactive compounds dilute the activity of the active compound.

In the practice of encoded split-pool methods physical separation of the solid support is required to accomplish two tasks: first, to physically isolate the individual library member after screening and, second, to de-code the identity of the tag and thus deduce the chemical structure of the member. A disadvantage of the chemically encoded split-pool methods is that the decoding is a linear, time-consuming process that cannot be effectively performed in a cost-effective manner for more than tens of thousands of compounds. Another disadvantage of the chemically encoded split-pool methods is that because compounds are synthesized such that there is a unique compound on each bead, the bead size limits the amount of each compound that can be made and isolated n a unique location. This limit on the quantity of the compound limits the bioassays that can be performed, and necessitates time consuming resynthesis of compounds for retesting. The use of larger beads causes problems with kinetics of movement of reagents into and out of the beads. A further disadvantage of chemically encoded synthesis is that placement of one bead per well of a multi-well plate is a highly inefficient process that is not currently scaleable to more then tens of thousands of compounds. This difficulty of arraying one bead per well results in the common practice of arraying many beads per well, which has similar disadvantages as the deconvolution method. Another disadvantage of the chemically encoded split-pool methods is that chemical tags introduce potential side reactions, and often react to a greater extend with the compound being synthesized than with the solid support resin, although the total quantity of the compound consumed is generally low.

In practice, both categories of split-pool methods require a large excess of solid support beads to ensure with reasonable certainty (99% confidence level) that all possible oligomers are made when a random split-pool strategy is employed. This is necessary because the exact identity of each bead (i.e. the identity of each oligomer) is lost due to the unstructured nature of the split-pool method. This presents a significant problem when scaling up these methods for the production of micromole or larger amounts of individual oligomers in the library.

Additional methods of encoding split-pool libraries include radiofrequency tagging or barcoding containers of solid support resin, described by Nicolaou et al., Radio Frequency Encoded Combinatorial Chemistry, ANGEW. CHEM. INT. ED. ENGL. (1995) 34(20), 2289; Armstrong et al., Microchip Encoded Combinatorial Libraries: Generation of a Spatially Encoded Library from a Pool Synthesis, CHIMIA 50 (1996), 258-60; and Xiao et al., Combinatorial Chemistry with Laser Optical Encoding, ANGEW. CHEM. INT. ED. ENGL. (1997) 36(7), 780-2. In these methods a machine is used to Rf tag or barcode each container of solid support resin after a synthetic step has been performed, thereby recording the reaction history of each container. Advantages of this method include the ability to quickly know the identity of the contents of each container for tens of thousands compounds and to make flexible quantities of each compound. A primary disadvantage to this method is the extremely high cost of the containers and the machines used to encode and sort the containers. Another disadvantage to this method is that the sorting process is linear, such that the Rf tag or barcode is individually read for each container, and then the container is directed into an appropriate bin. This linear sorting process currently takes approximately 10 hours for 10,000 compounds and is limited to a maximum capacity of 100,000 compounds, which would take 4 days.

A one dimensional method for spatially-addressable combinatorial synthesis, in which solid supports are arranged in a line has been described by Furka et al., String Synthesis. A Spatially Addressable Split Procedure, JOURNAL OF COMBINATORIAL CHEMISTRY (2000) 2, 220-223; Furka et al., Redistribution in Combinatorial Synthesis. A Theoretical Approach, COMBINATORIAL CHEMISTRY AND HIGH THROUGHPUT SCREENING (2000) 3, 197-209. Furka et al. strung Mimotopes “crowns” (solid supports) on pieces of fishing line, tied the ends of the fishing lines together, and submitted the solid supports to the reaction conditions for a step of the synthesis. To reorganize the solid supports in preparation for the next reaction, Furka et al. would move the solid supports from one piece of fishing line onto a different piece of fishing line in a defined manner. A drawback of this procedure is that it would be extremely difficult to automate or scale up the method to handle hundreds of thousands or millions of solid supports in this manner. In practice, only 125 solid supports were used in the method disclosed by Furka et al.

W097/35198 describes a method for the synthesis of a spatially-dispersed combinatorial library of oligomers, in which the oligomers are distributed in a controlled manner. These oligomers are comprised of a series of monomers which are introduced into the oligomers in a sequential and stepwise fashion via chemical transformation steps (hereafter referred to as “steps”). These monomers are comprised of subsets of monomers such that the first subset of monomers is introduced in the first step, the second set of monomers is introduced in the second step, etc. The method further describes a means for introducing the monomers in a sequential and stepwise fashion on a series of solid supports. The number of supports equals the number of oligomers in the library. The publication also recites that supports are arranged in, and subsequently redistributed in a controlled manner between a series of arrays that provide a means for holding the supports in physically discrete locations such that the exact identity of each support is provided for each location. A further aspect of the method described in W097/35198 is that the redistribution of supports is carried out in a controlled fashion between each step such that all possible combinations of possible oligomers are synthesized. Further, the publication recites that the positions of the supports are known during the synthesis of the experiment such that the identity of the oligomer is unequivocally established by its location. The applied method recited achieves a geometric amplification in the number of library members synthesized relative to the number of synthetic steps required while providing individual library members in a spatially-dispersed format.

The apparatus used in the method recited in W097/35198 retains the solid supports in place in an array within a single reaction vessel and then permits moving of the solid supports in a defined manner to new arrays for subsequent steps. This has limited applicability with regard to permitting lateral movement of the supports within an array since the solid supports rest in fixed slots in an array rack. As such, the solid supports need to be lifted out of the rack or holder and placed in a new rack in order to “shuffle” the solid supports between reactions. The robotics necessary to shuffle hundreds of thousands or millions of solid supports in the manner described in W097/35198 would be extremely expensive, and would either require machining to extremely high tolerances or would be prone to error, particularly in terms of the dropping of solid supports or missing the opening when trying to insert the solid support into the reaction vessel. The shuffling process for hundreds of thousands or millions of solid supports in the manner described in W097/35198 would be extremely time consuming.

There is thus a need in the combinatorial chemistry art for a technique which can achieve geometric amplification in the number of library members synthesized relative to the number of synthetic steps required and which holds the solid supports in place while simultaneously allowing the solid supports to be easily moved in a lateral direction so as to conveniently, efficiently, affordably and scalably “shuffle” the solid supports between reactions.

### SUMMARY OF THE INVENTION

The present invention provides an apparatus and method by which solid supports may be retained in place in an array within an reaction vessel, but which allows easy lateral manipulation of the solid supports in a defined manner onto new arrays for subsequent steps in the synthesis of combinatorial chemistry libraries of compounds.

According to the present invention, a pair of racks, retainers or support carriers for holding solid supports is provided in the configuration of a solid support carrier having a plurality of “fingers”, tines or rods extending from one end and between which longitudinal channels or spaces are defined. In accordance with one embodiment of the present invention, a first solid support carrier is positioned on top a second solid support carrier such that the tines criss-cross with each other (i.e. the solid support carriers are oriented 90° to one another) to define longitudinal channels within which the solid supports are positioned and retained. When the solid supports are positioned and retained on a single solid support carrier, the longitudinal channels support the solid supports thereon but permit free lateral movement along the length of the longitudinal channel so that the solid supports may be easily and efficiently “shuffled” between reactions.

More particularly, when two solid support carriers are aligned and interfaced with one another, the solid supports positioned thereon cannot move substantially relative to one another or slide off the solid support carrier. An alternative method of preventing the movement of the solid supports on a solid support carrier or solid support carrier is to use a barrier that is positioned at the end of a single solid support carrier, or to apply a force upon the solid supports positioned on the solid support carrier such that the solid supports cannot move substantially to one another or slide off of the solid support carrier. The barrier used in this embodiment could include, but is not limited to, an object positioned at the end of the rods and which blocks the path of the beads or spheres from being removed or falling off of the solid support carrier. The force applied to retain the beads or spheres on the rods may include, but is not limited to a magnet, gravity or frictional force. This alternative embodiment of retaining the solid supports on a single solid support carrier using a barrier or force should be understood to be equivalent to the interfacing of two solid support carriers in order to substantially support, retain and immobilize the solid supports thereon.

It should be understood that solid support carriers in accordance with the present invention, are not limited to the solid support carriers as described hereinabove.

Any carrier which possesses the properties of allowing one or more solid supports to move in channels or to be retained and substantially immobilized thereon is intended to be encompassed the present invention. For example, a corrugated porous surface would serve the same function as the solid support carrier described.

Each pair of solid support carriers and the solid supports associated therewith typically interface with a single reaction vessel. In the embodiment that comprises two aligned and interfacing solid support carriers, when one of the two intersecting solid support carriers is removed, the solid supports can slide laterally in one dimension, in the channel defined between the tines within the plane of the solid support carrier. However, during this movement, the solid supports in a channel will maintain their positions relative to one another and the knowledge of the relative positions of all solid supports will be maintained so that their identity will be traceable based on their location. Removing one of the solid support carriers allows the solid supports to be moved onto the other solid support carrier easily and in a manner which keeps the solid supports spatially arrayed. A typical shuffling (rearray) process involves sliding the solid supports from each row of a solid support carrier (within a channel) onto a new receiving solid support carrier and repeating the process for each solid support carrier full of solid supports, until all the new receiving solid support carriers have one row each of solid supports from each of the previous sets of solid support carriers. After all the solid supports have been moved onto new solid support carriers in the desired manner, the second intersecting solid support carrier is replaced so that the solid supports are once again immobile. To shuffle the solid supports in a second dimension, the second solid support carrier is removed, which was perpendicular to the solid support carrier which was removed in the first shuffling. In this manner, the solid supports can be shuffled in a second dimension, such that the solid supports which were previously moved in unison as rows, can now be moved onto different solid support carriers by shifting the solid supports by columns.

In the embodiment in which a barrier is used or a force is applied to retain and immobilize the solid supports on a single solid support carrier during reactions, the barrier or force is removed, which allows the solid supports to be shuffled in one dimension. The barrier or force is then replaced on the single solid support carrier in order to again support, retain and immobilize the solid supports positioned thereon. To shuffle the solid supports in a second dimension (e.g. columns instead of rows), the barrier or force is removed and a second solid support carrier is interfaced (engaged) with the first solid support carrier. The first solid support carrier is then removed from the interfacing alignment with the second solid support carrier such that the solid supports now rest in the channels on the second solid support carrier. As used herein, the word “barrier” includes forces such as gravity, friction and magnetism. In an alternative to engaging the second solid support carrier with the first solid support carrier prior to removal of the first solid support carrier, instead, the first solid support carrier may be removed in such a manner that the solid supports retain their positions. The second solid support carrier could then be engaged after the first solid support carrier is removed.

It is not required that entire rows and/or columns be moved together. Portions of rows and/or columns can be moved onto new solid support carriers apart from the rest of the rows and/or columns. Alternatively, multiple rows and/or columns at a time can be moved onto new solid support carriers and later, in subsequent steps, these rows and/or columns can be moved individually onto new solid support carriers, thus facilitating the synthesis of libraries of compounds with more than three diversity positions.

Reactions can be performed on these solid supports wherein all the solid supports held in a set of intersecting solid support carriers or on a single solid support carrier are subjected to a chemical reaction in a single reaction vessel. In one embodiment of the present invention, both the solid support carrier(s) and the solid supports positioned thereon are immersed in a reaction vessel. In an alternative embodiment, only the solid supports are immersed in a reaction vessel. In comparison to parallel synthesis techniques, the embodiments of the present invention have the advantage of reducing the total number of reaction vessels needed from being equal to the number of solid supports to instead being equal only to the number of building blocks used in any given step. The embodiments of the present invention also provide the advantage of substantially facilitating the redistribution of the solid supports. For example, the redistribution process for one million compounds comprising three sets of one hundred building blocks each could be accomplished in only one hundred movements of solid supports, where all one hundred solid supports from a row/column of each of the one hundred solid support carriers are moved at once. Then another set of rows/columns of one hundred solid supports from each of the one hundred solid support carriers are moved at once, etc.

Throughout the application, the terms “redistribution”, “shuffling”, “arraying” and “rearraying” have been used interchangeably and are intended to mean the same thing.

The above description sets forth rather broadly the more important features of the present invention in order that the detailed description thereof that follows may be understood, and in order that the present contributions to the art may be better appreciated. Other objects and features of the present invention will become apparent from the following detailed description considered in conjunction with the accompanying drawings. It is to be understood, however, that the drawings are designed solely for the purposes of illustration and not as a definition of the limits of the invention, for which reference should be made to the appended claims.

The details of one or more embodiments of the invention are set forth in the accompanying description below. Although any methods and materials similar or equivalent to those described herein can be used in the practice or testing of the present invention, the preferred methods and materials are now described. Other features, objects, and advantages of the invention will be apparent from the description. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference.

### DETAILED DESCRIPTION OF THE DRAWINGS

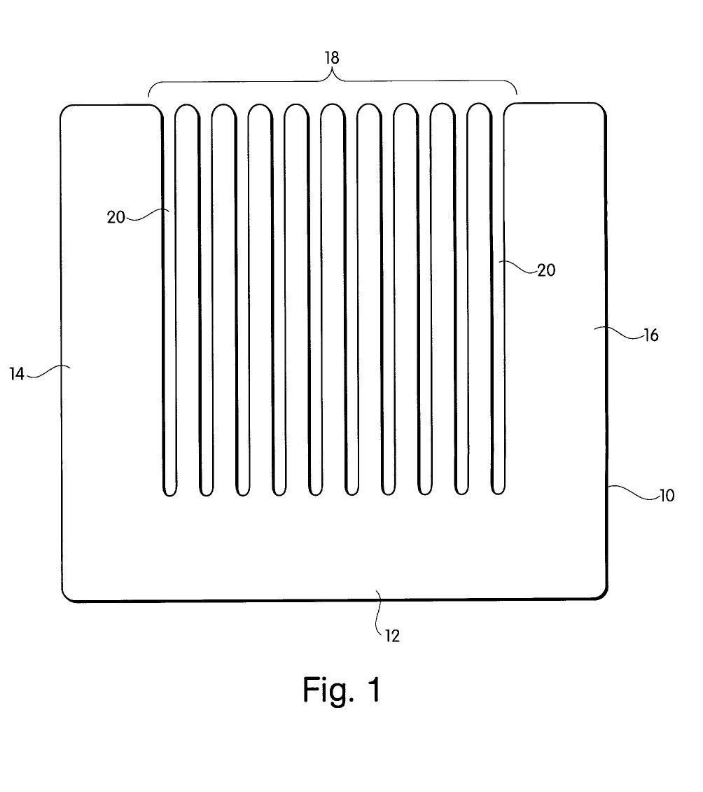

FIG. 1 is a top plan view of a single solid support carrier in accordance with one embodiment of the present invention;

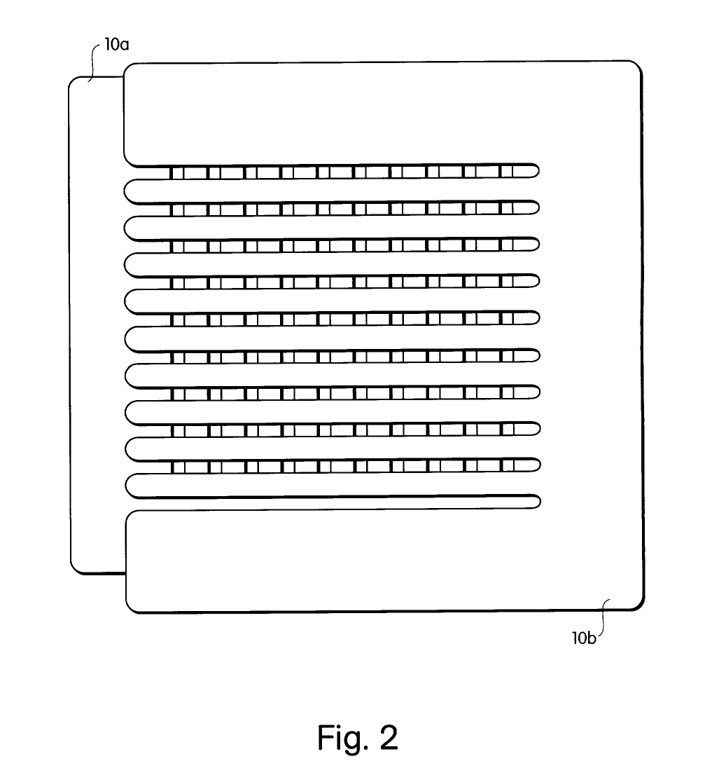

FIG. 2 is a top plan view of a pair of solid support carriers in accordance with one embodiment of the present invention with the solid support carriers superimposed upon each another and oriented 90° to one another;

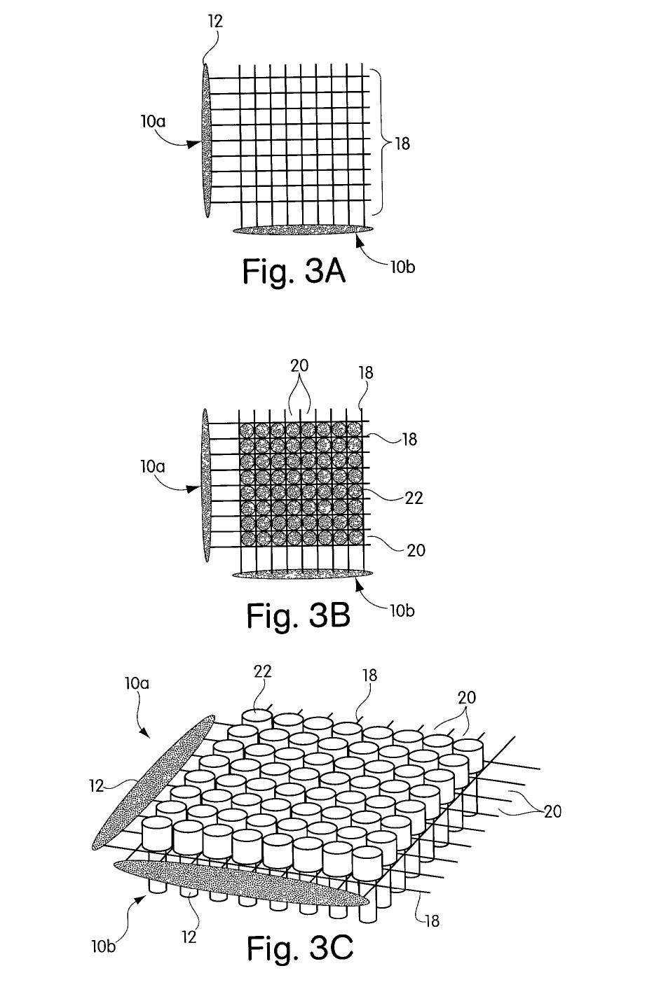

FIG. 3A is a top plan view of an alternative embodiment of the present invention illustrating two aligned and intersecting solid support carriers;

FIG. 3B is a top plan view of the embodiment illustrated in FIG. 3A having a plurality of solid supports positioned thereon;

FIG. 3C is a perspective view of the embodiment illustrated in FIG. 3B;

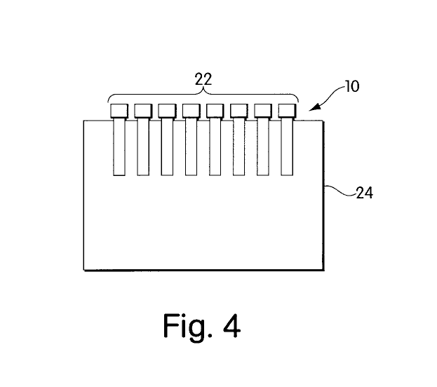

FIG. 4 is a side view illustrating a plurality of solid supports in one channel of a solid support carrier and which are spatially addressed in 2-dimensions within a reaction vessel within a reaction vessel;

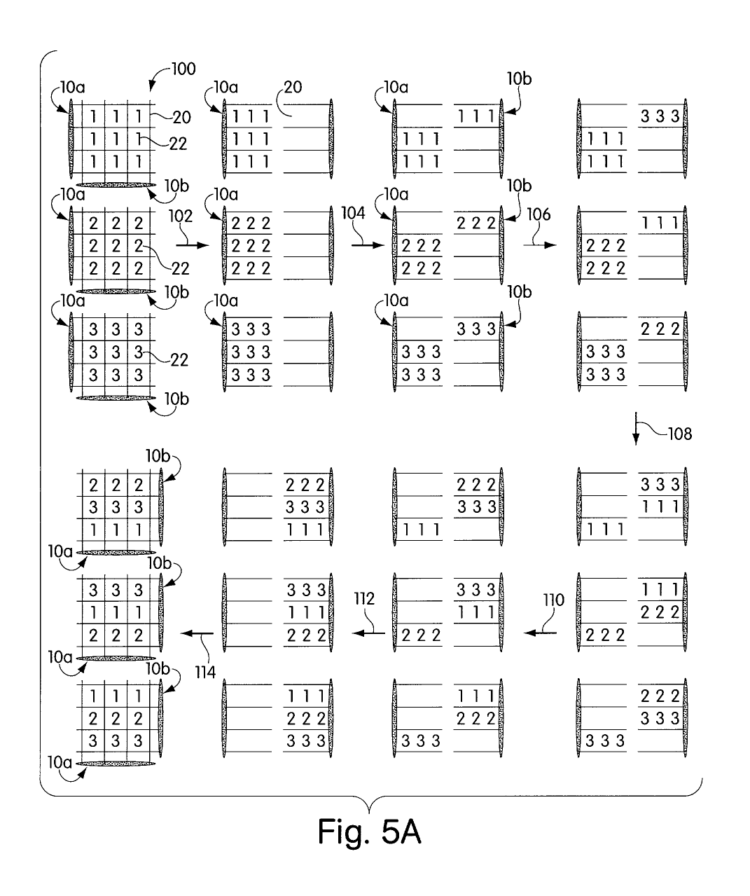

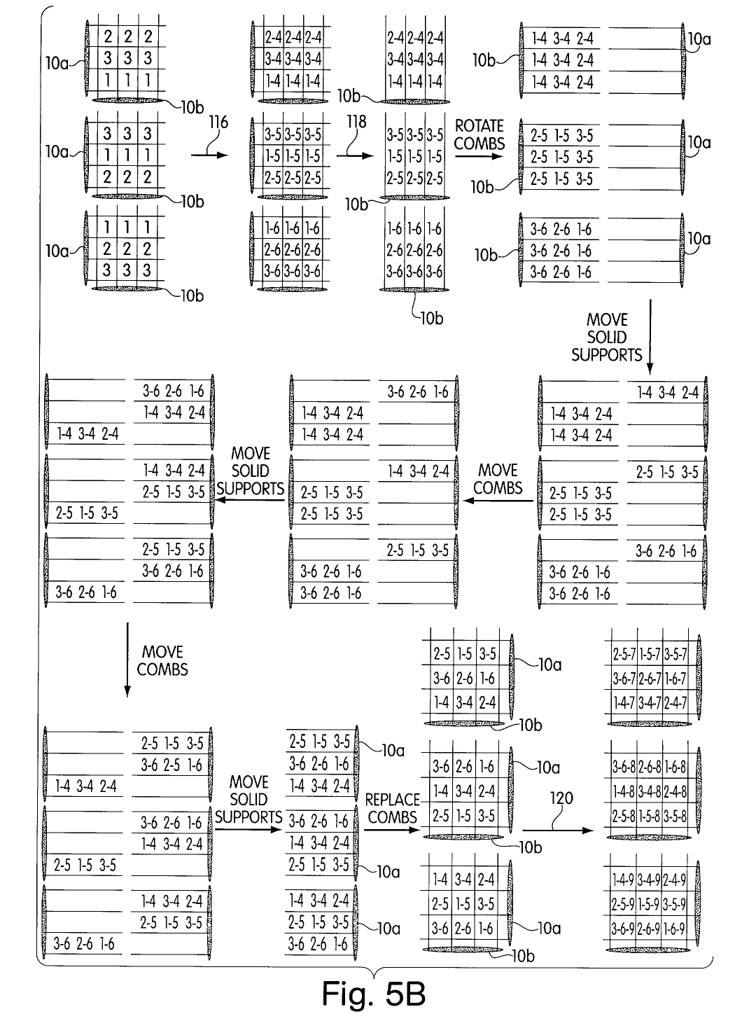

FIGS. 5A and 5B illustrate an alternative flow chart demonstrating the movement of solid supports as illustrated in FIGS. 5A and 5B;

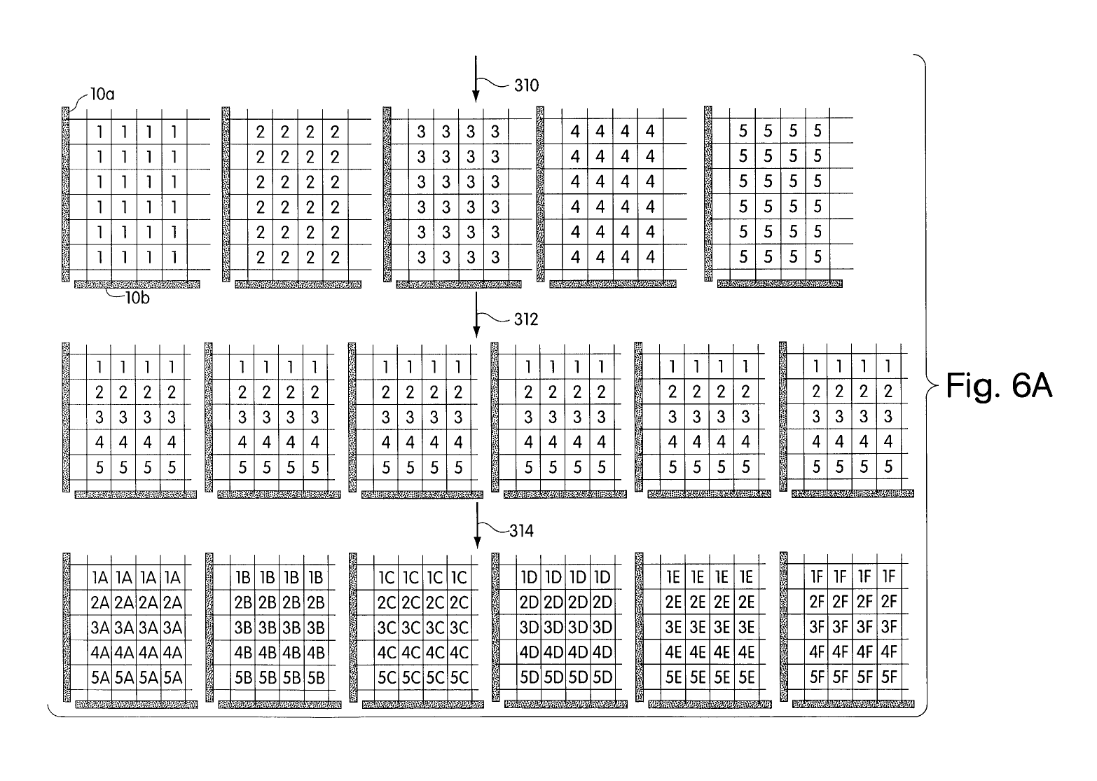

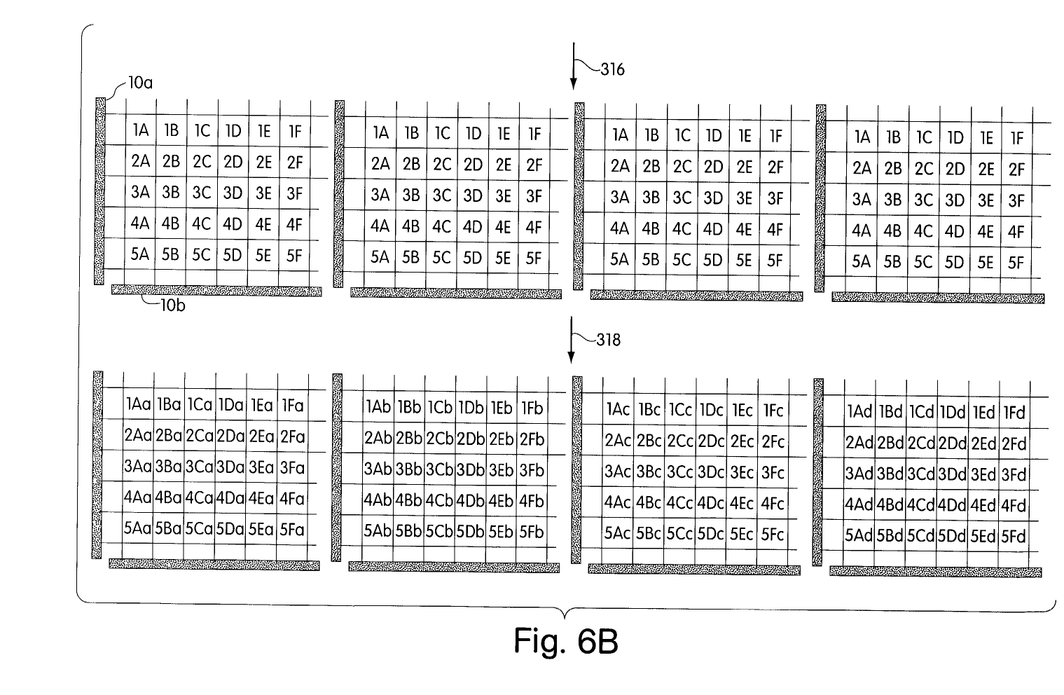

FIGS. 6A and 6B illustrate a flow chart demonstrating an alternative embodiment of movement of solid supports containing different building blocks when building a combinatorial library of compounds, wherein the number of building blocks (or more generally, chemical modifications) in the three steps is 5, 6, and 4;

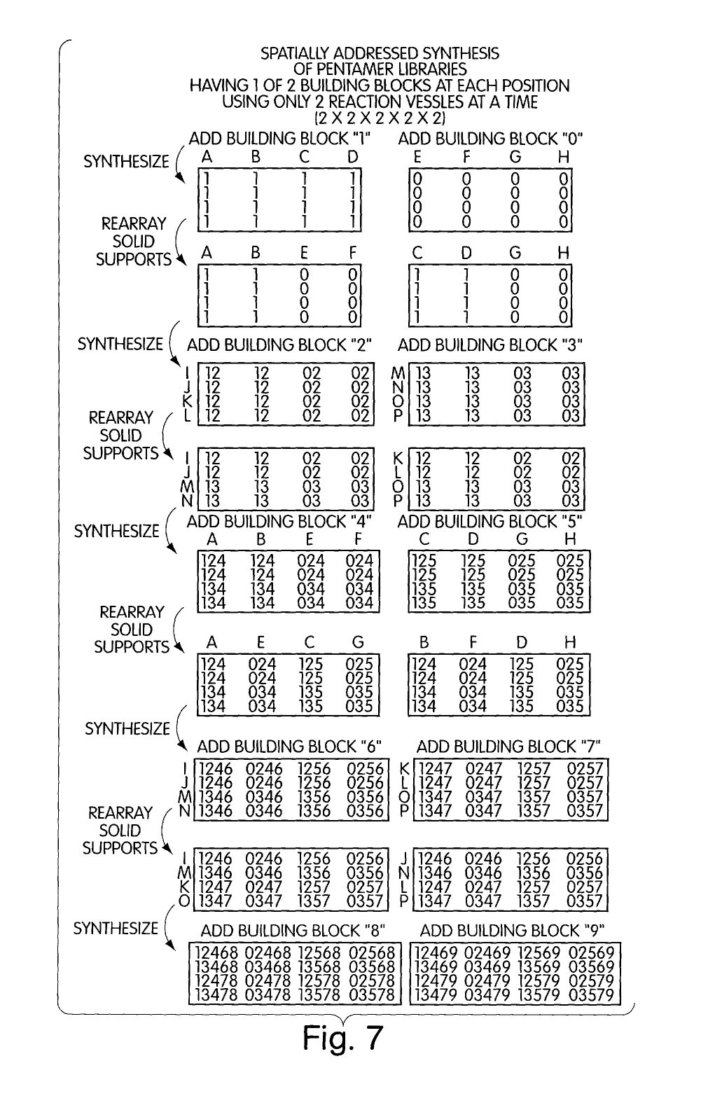

FIG. 7 is a flow chart demonstrating the movement of solid supports as illustrated in FIGS. 3-6 and illustrating a library made with five steps, using two building blocks or chemical modifications per step;

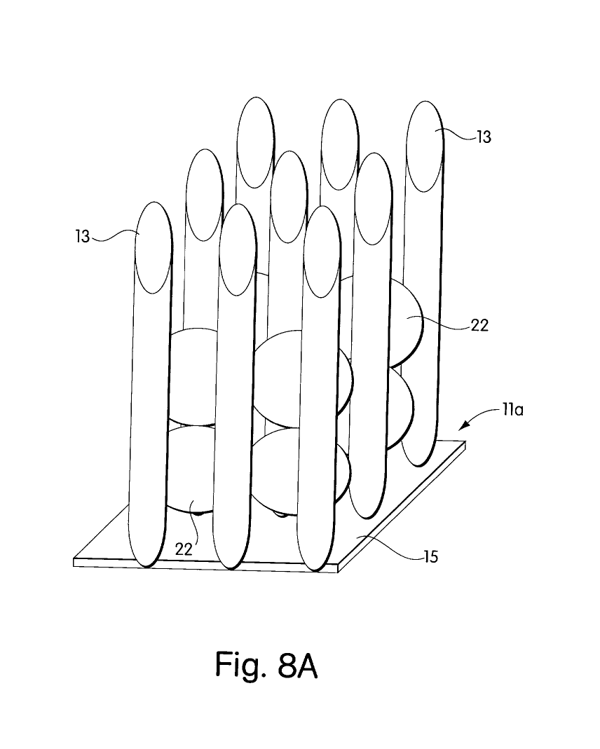

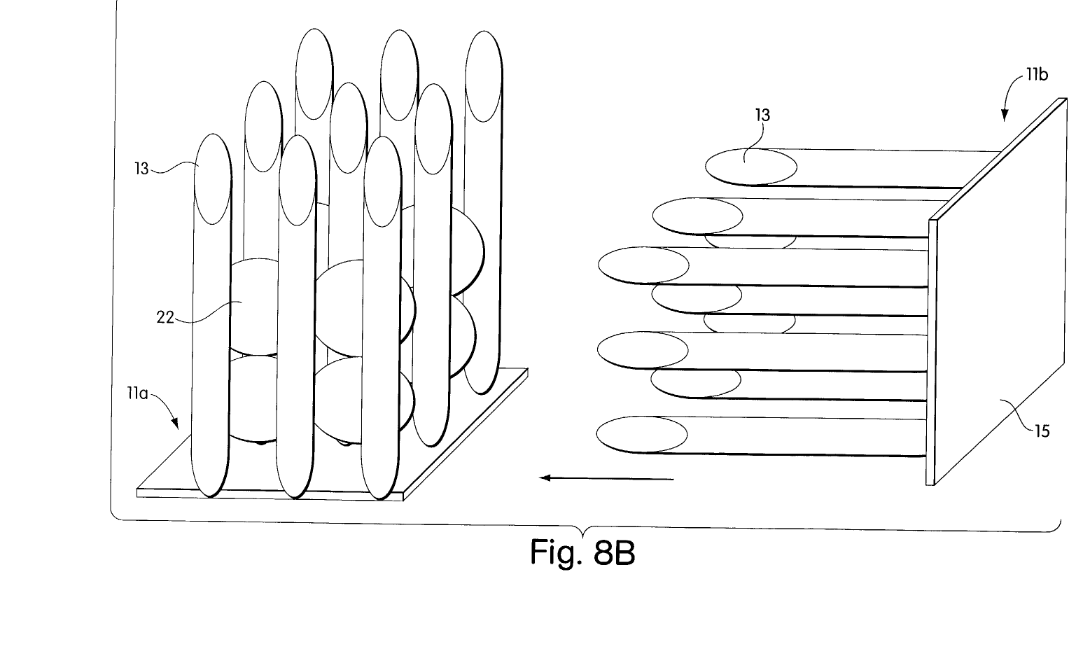

FIG. 8A is a front-perspective view of a three-dimensional embodiment of the solid supports and solid support carrier in accordance with the present invention;

FIG. 8B is perspective view of the embodiment illustrated in FIG. 8A and illustrating the positioning of a second support carrier in relation to the first support carrier for retaining solid supports; and

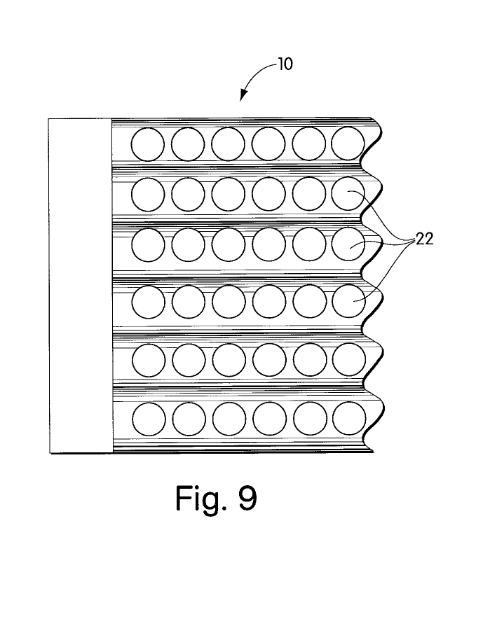

FIG. 9 is a top plan view of solid supports and a single solid support carrier in accordance with one embodiment of the present invention.

### DETAILED DESCRIPTION OF THE INVENTION

FIG. 1 illustrates a preferred embodiment of the apparatus of the present invention. In this embodiment, a solid support carrier 10 is configured in the form of a “comb” having a periphery defined by a first end 12 and having first and second sidewalls 14, 16 which extend therefrom to facilitate grasping and for easy manipulation. A plurality of elongated finger-like protrusions or tines 18 extend from first end 12. FIG. 2 illustrates a first solid support carrier 10a and a second solid support carrier 10b, wherein first solid support carrier 10a and second solid support carrier are aligned and interfaced with one another such that the tines 18 of each respective solid support carrier are oriented substantially perpendicular to each other.

Sidewalls 14, 16 of solid support carrier 10 may also function as tines and as illustrated in the embodiment shown in FIG. 3A, may be less defined than as illustrated in the embodiment in FIG. 1. Between the plurality of tines 18, a corresponding plurality of elongated channels 20 are formed so that there exists an alternating arrangement of tines and channels. The number of tines 18 and corresponding channels defined on each solid support carrier 10 is determined by the parameters of the experiment or procedure in which solid support carriers 10 are to be used. It should be understood that the solid support carriers 10 are not limited to the configuration of a comb, and any carrier capable of supporting and retaining solid supports thereon is contemplated by the present invention. Additionally, the invention is in no way limited by the number of times and channels which may be defined on each solid support carrier.

Channels 20 are advantageously configured to receive one or a plurality of solid supports 22 (shown in FIGS. 3B and 3C) which are slidably positioned therein. Solid supports 22 positioned in channels 20 are supported by tines 18 on each side of the solid support 22 and which define the channel 20 defined between the tines 18. Solid supports 22 are generally known in the art as any material, or solid support combination of materials, having a rigid or semi-rigid surface and having functional groups or linkers, or that is capable of being chemically derivatized with functional groups or linkers, that are suitable for carrying out chemical synthesis reactions. Such materials will preferably take the forms of, but are not are not limited to, various shapes of polymers, including polystyrene, grafted co-polymers of combinations of polystyrene (optionally cross-linked with divinylbenzene), polypropylene and/or polyethylene glycol or substituted variants thereof, as well as combinations thereof. Furthermore, since solid supports 22 may exist in a variety of sizes, the invention is not limited with respect to the size of the channels 20, which may be of any size to accommodate one or more solid supports 22 therein. Additionally, individual reaction vessels or vials may take the place of solid supports. As recited herein, a linker is a moiety, molecule, or group of molecules attached to a solid support and spacing a synthesized oligomer or compound from the solid support.

FIGS. 3A and 3B are simplified illustrations of the solid support carriers 10 shown in the configuration used in accordance with the present invention. As illustrated, solid support carrier 10 are aligned or interfaced and superimposed one upon the other. When solid support carrier 10 are aligned with each other and superimposed one upon the other in accordance with the present invention, one or more solid supports 22 may be positioned in one or more channels 20 defined between the tines 18 of the solid support carrier 10. As illustrated in FIGS. 3B and 3C, when both solid support carrier 10a and 10b interface with each other, solid supports 22 are supported thereon and movement is restricted. In practice, one or a plurality of solid supports 22 are initially positioned within channels 20 of a first solid support carrier, such as 10a. A second solid support carrier 10b is then oriented, aligned and interfaced with the first solid support carrier 10a and positioned relative to the first solid support carrier 10a as illustrated in FIG. 3B so that solid supports 22 are supported and retained by the of tines 18 of the solid support carriers 10a, 10b. In another embodiment, second solid support carrier 10b is oriented substantially perpendicular to first solid support carrier 10a when first solid support carrier 10a and second solid support carrier 10b are aligned and interfaced with one another. As used herein, the term “substantially perpendicular” is intended to be an angle of approximately 90°. However, the invention is not limited in this respect and the solid support carriers may be oriented at angles both larger and smaller than 90°.

In an alternative embodiment (not shown), solid supports 22 may be retained on a single solid support carrier 10 and immobilized thereon by a barrier or force, which is applied to the solid supports. This embodiment of retaining the solid supports on a single solid support carrier using a barrier or force should be understood to be equivalent to the interfacing of a first and second solid support carrier, such as 10a and 10b illustrated in FIGS. 3B and 3C, in order to retain and immobilize the solid supports thereon.

FIG. 3C illustrates an embodiment of the present invention illustrating an 8×8 array of solid supports positioned and supported by two solid support carriers 10a, 10b oriented at 90° in accordance with the present invention. When one of the solid support carriers 10a or 10b are removed, solid supports 22 are easily slidable laterally within their respective channels 20. It is understood that the 8×8 array illustrated in FIG. 3C is illustrative only and the invention is not limited in this respect. In practice, arrays can be as large as desired. In practice, there are generally numerous reaction vessels and numerous corresponding sets of solid support carriers associated therewith such that there is typically one reaction vessel and one corresponding set of solid support carriers per building block (or any chemical modification).

FIG. 4 provides a side-view of a plurality of solid supports 22 positioned within a channel 20 of one solid support carrier 10 which is positioned in a vessel 24 in which reactions are carried out upon the solid supports 22. When solid supports 22 are positioned within channels 20 defined between tines 18 of a solid support carrier, and supported thereon, the solid supports are held in a spatially addressed manner during chemical synthesis and may be redistributed and/or rearrayed along the tines 18 of the solid support carrier 10.

FIG. 5A is a flow chart which demonstrates the step-by-step positioning and rearrangement of a first solid support carrier 10a and a second solid support carrier 10b, and solid supports 22 containing different building blocks, which are positioned and supported on the solid support carrier in the course of building a combinatorial library/collection of compounds. In this embodiment as illustrated in FIG. 5A, first solid support carrier 10a and second solid support carrier 10b are aligned and interfaced with one another. For simplicity, only steps involving attachment of a set of building blocks or performance of a set of chemical modifications are shown. As many other reactions as desired, such as reactions in which the same set of conditions are used on all the compounds on all the sets of solid support carriers, may be performed before or after the illustrated steps.

As indicated by 100 in FIG. 5A, a first solid support carrier 10a and a second solid support carrier 10b are aligned and interfaced with each other and superimposed upon one another to define an array (indicated as 100) consisting of three grids. Although first solid support carrier 10a and second solid support carrier 10b appear in FIG. 5A to interface substantially perpendicular to one another, the invention is not limited in this respect. For simplifying the illustration of the method of using the solid support carriers 10a, 10b of the present invention, inside these grids are shown nine sets of three of the numerals 1-9, one set of numerals per grid. The different numerals represent different building blocks or chemical compounds, the hyphens represent connectivity between the building blocks or compounds, and each set of numerals on one solid support 22. As such, the array illustrated in 100 in FIG. 5A shows 27 solid supports, arrayed in 3 grids/solid support carrier of 3 by 3. The invention is in no way limited to this grid size, and this number is used only to be small enough to provide a detailed demonstration in the figure. It should be understood that because there are three different compounds present, by implication, one step may already have been performed on the solid support in which three different chemical modifications were introduced, or three different compounds were attached to the solid support.

The arrow indicated by 102 in FIG. 5A indicates the removal of one of either the first or second solid support carrier 10a, 10b. Without being limited thereby, the remainder of the description of FIG. 5A will proceed as if second solid support carrier 10b is the solid support carrier removed in the step indicated by 102. The removal of second solid support carrier 10b affords the solid supports 22 (indicated by the numerals) the capability of sliding laterally within the channels 20 of first solid support carrier 10a. The solid supports on first solid support carrier 10a are then “rearrayed” onto second solid support carrier 10b, which has been positioned to receive the solid supports from first solid support carrier 10a. As indicated by arrow 104, one row of solid supports 22 from each first solid support carrier 10a is slid onto the corresponding receiving row of second solid support carrier 10b. Next, as indicated by 106, the receiving second solid support carriers 10b are moved so that the receiving second solid support carriers 10b which received solid supports with one compound will now receive solid supports 22 with another chemical compound. Next, as indicated by 108, the second row of solid supports is moved onto the receiving second solid support carrier 10b. As indicated by 110, the receiving second solid support carrier 10 are then moved again so that each receiving second solid support carrier 10b will be in position to receive a set of solid supports 22 it has not yet received. The third row of solid supports 22 is then slid onto the receiving second solid support carrier 10b as indicated at 112. Each of the first solid support carriers 10a removed in step 102 are then “replaced” or interfaced with the second solid support carrier 10b so that the solid supports 22 again completely supported by the tines 18 of the first and second solid support carriers 10a, 10b (indicated by arrow 114).

FIG. 5B is a continuation of the process commenced in FIG. 5A. FIG. 5B, commences with the next synthetic step in the process, indicated by arrow 116, in which building blocks (compounds) are introduced. The hyphens indicate connectivity between the first three compounds and the new building blocks, represented by the numerals 4, 5, and 6, which have been attached to the solid supports containing the first three compounds or chemical modifications which have been performed on the first three compounds. In FIG. 5B, the process described in FIG. 5A, whereby the solid supports are moved onto new solid support carriers such that they are distributed among the new solid support carriers, is repeated. In FIG. 5B, however, solid support carrier 10a is first removed (indicated by arrow 118) and the solid supports 22 remain upon second solid support carrier 10b. In this manner, columns are redistributed instead of rows (or vice versa). In the final, third synthetic step indicated by arrow 120, the three building blocks or chemical modifications are represented by the numerals 7, 8 and 9, and now there are 27 different entities.

FIGS. 6A and 6B show a flow chart illustrating a process similar to that described in FIGS. 5A and 5B, and which illustrates that the shuffling and rearrangement of the solid support carriers and the solid supports positioned and retained thereon is not limited to the formation of chemical libraries/collections in which the number of building blocks is the same from one step in the process to the next. The detailed steps showing how the redistribution proceeds is omitted, since they have been previously shown with regard to FIGS. 5A and 5B, and since the steps do not differ materially. FIGS. 6A and 6B illustrate an alternative embodiment which uses 5, 6 and then 4 building blocks in the three steps of the synthesis. Once again these numbers are not meant to be limiting, but are simply for the purpose of illustrating the method of the present invention and that different numbers of building block or chemical modifications can be used in the various steps throughout the synthesis. Once again, for simplicity, only steps involving attachment of a set of building blocks or performance of a set of chemical modifications are shown. As many other reactions as desired, such as reactions in which the same set of conditions are used on all the compounds on all the sets of solid support carriers, may be performed before or after the indicated steps.

In the first step in FIG. 6A indicated by arrow 310, the action of attaching five different building blocks, or making five different chemical modifications to the chemical compound or building blocks already on the solid support takes place. In the first row of FIG. 6A, each of the five arrays is comprised of first solid support carrier 10a and a second solid supports carrier 10b aligned and intersecting with one another, and having the solid supports (indicated by the numerals) containing different building blocks, positioned and supported on the solid support carrier. The numerals 1-5 represent the different building blocks or chemical modifications which are used. The next step indicated by arrow 312 indicates the rearraying of the solid supports in an orderly manner to redistribute them onto solid support carriers in preparation for the next step where different building blocks or chemical modifications are used.

The next step indicated by arrow 314 refers to the attachment of the next set of building blocks or the performance of the next series of chemical modifications. The upper case letters, A, B, C, D, E and F, in the six boxes represent six different building blocks added to, or chemical modifications of, the different compounds originally represented by the numbers 1-5. In the next rearraying step indicated by arrow 316, the solid supports are redistributed by columns as shown. In the final synthesis step indicated by arrow 318, four different building blocks or chemical modifications are used, and which are represented by the lower case letters, a, b, c, and d.

FIG. 7 is a flow chart similar to FIG. 6. FIG. 7 is provided to facilitate the understanding that method of the present invention is not limited to cases where the number of steps with multiple building blocks is limited to just three or four steps. On the contrary, any number of steps with various numbers of building blocks can be accommodated. FIG. 7 shows a library (i.e., a collection of chemical compounds) made with five steps, using two building blocks or chemical modifications per step. These numbers could be arbitrarily large, and will generally be much greater in practice. The number of building blocks or chemical modifications is only used here for the purpose of providing a detailed demonstration in the figure and is in no way meant to be limiting. The number of steps (five) is also used so as to be small enough to provide a detailed demonstration in the figure and is in no way meant to be limiting. All numbers (of building blocks, chemical modifications and steps) would typically be much greater in practice or in the performance of a set of chemical modifications are shown. As many other reactions as desired, such as reactions in which the same set of conditions are used on all the compounds on all the sets of solid support carriers, may be performed before or after the indicated steps. The two boxes shown at the top of FIG. 7 represent two reaction vessels. The 4 by 4 grids of numbers represent solid supports on which compounds are synthesized. The arrow with the word “synthesize” beside it and the words “add building block ‘1’” and “add building block ‘0’” represent the action of attaching two different building blocks to the solid supports or to the compounds which are on the solid supports. The arrow with the word “rearray” beside it represents the action of moving the solid supports from the solid support carriers they were in the top two sets of boxes to the solid support carriers they are in the next set of boxes below in such a way that the solid supports are moved to the positions shown.

The way in which this process would be carried out is to remove one of the solid support carriers and slide the solid supports onto the next set of solid support carriers. In the example depicted in FIG. 7, columns are moved, and column A is moved with B, C with D, E with F, and G with H. The next (third) arrow down with the word “synthesize” beside it and the words “add building block ‘2’” and “add building block ‘3’” represent the action of attaching two different building blocks to the two different compounds on the solid supports, or more generally, chemically modifying the compounds on the solid supports in two different ways. For the case of synthesis of oligomers of repeating units of the same building blocks, “2” and “3” could be the same as “1” and “0.” The next (fourth) arrow down with the word “rearray” beside it represents the action on moving the solid supports from the solid support carriers onto new solid support carriers in an orderly way. In this example, rows are moved. Row I is moved with J, K with L, M with N, and 0 with P. Continuing down, the next synthesis step is to add building blocks (or more generally, different chemical modifications) “4” and “5” to the two different reaction vessels represented. For the next “rearraying” step, columns A and B, which were previously moved together, into the same new solid support carrier to provide extra copies to later be split apart, are now moved separately into different solid support carriers. The same is true with C and D, E and F, and G and H. In the next synthesis step building blocks “6” and “7” are added, or as is true throughout, more generally, the compounds are modified in two different ways. In the next “rearraying” step, rows I and J, which were previously moved together to the same new solid support carrier to provide extra copies for later separate modifications, are now spilt apart and moved to different solid support carriers. The same is true for K and L, M and N, and 0 and P. Finally the last building blocks (or more generally, different chemical modifications) “8” and “9” are added.

It is intended to be clear to those skilled in the art, that the techniques described herein can be extended to synthesize combinatorial libraries in which more than three combinatorial steps are employed. A computer algorithm can be designed which takes as input the goals of a synthetic experiment namely, the desired number of combinatorial steps and the desired number of monomers used in each combinatorial step. The algorithm can then generate a map of the protocol required to satisfy the experimental goal. This map would contain the same information as that provided in the figures used herein.

The embodiments described above are directed generally to methods for synthesizing libraries of compounds of very modest size. The process is readily extrapolated to the synthesis of much larger libraries. As such, to more clearly illustrate how the apparatus of the present invention and method of procedure in which it is used makes feasible the synthesis of much larger collections of compounds in a spatially addressed manner than was previously achievable, the following example describes the synthesis of 1,000,000 compounds from three sets of building blocks with 100 building blocks in each set. An example is described where the number of sets of building blocks is three and the number of building blocks in each set are all 100. One million solid supports will be hung from the 100 solid support carriers. A solid support carrier will be interfaced and aligned substantially perpendicular (oriented at approximately 90°) to the first solid support carrier in such a way as to retain the solid supports in place in the channels defined by the tines of the solid support carriers. In this embodiment, a grid is used, where the solid supports are arrayed in rows and columns. Each of the 100 sets of solid support carriers will hold an array which has 100 rows and 100 columns of solid supports. Based on a protocol, whatever necessary reactions are then performed on the solid supports. One of these reactions will introduce 100 distinct building blocks. Each building block will be introduced in a separate reaction vessel and hence each set of solid support carriers will hold solid supports with different compounds from one another. There will now be 10,000 copies each of a single compound on each of the 100 sets of solid support carriers, and the library will contain 100 distinct compounds. Any further reactions necessary in which the same reactants are used on all compounds will be performed.

Next, prior to introducing the second set of building blocks, the solid supports will be moved in an ordered manner such that representative compounds from each of the first set of building blocks will be present in each of the reaction vessels where the second set of building blocks is to be introduced, and such that the position of every compound is still known. The specific manner in which this is accomplished is to remove of the solid support carriers from each set of perpendicular interlocking solid support carriers so that the solid supports can slide along the channels of the solid support carrier on which they rest. The solid supports would then be transferred onto new solid support carriers in which each of the 100 rows of solid supports on each new solid support carrier would contain one row of solid supports from each of the previous 100 sets of solid support carriers. Sliding the solid supports from the channels of one solid support carrier to channels of the next solid support carrier is substantially facilitated by the solid support carrier in accordance with the present invention. In addition, the rows or columns of solid supports from all the solid support carriers can be moved in unison in parallel on to the solid support carrier receiving the solid supports. Therefore, in this example, 10,000 solid supports can be moved on to their receiving solid support carriers at once: 100 solid supports from each of the 100 solid support carriers. Therefore, only 100 movements of the solid supports would be required to redistribute all one million solid supports. The second solid support carrier would then be aligned and interfaced with the solid support carrier holding the solid supports so that the solid supports are retained in place on the solid support carriers. The rows would still be 100 solid supports across, and would thus still have 100 columns. At this point each set of solid support carriers contains 100 copies each of 100 different compounds. Any necessary reactions where the reactants used on each compound are the same would then be performed.

Next, reactions with the second set of 100 building blocks would be performed, with one building block per reaction vessel. There would now be 100 copies of 10,000 distinct compounds in the library. At this point each row of solid supports in each set of solid support carriers contains a different compound. All the solid supports within any given row contain the same compound. Any necessary reactions where the reactants used on each compound are the same will be performed. Prior to introducing the third set of building blocks, the solid supports will be moved in an orderly manner such that representative compounds from each of the first two sets of building blocks will be present in each of the reaction vessels where the third set of building blocks is to be introduced, and such that the position of every compound is still known. In this case, the manner in which that will be done is to remove the solid support carrier perpendicular to the solid support carrier removed previously. Solid supports will then be transferred in columns to new solid support carriers. Sliding the solid supports from within one channel of one solid support carrier to channels of the next solid support carrier facilitates this transfer. This ability to transfer 10,000 solid supports in a single movement is a significant improvement over prior art methods. The first and second solid support carrier would then be interfaced and aligned substantially perpendicularly to one another so that the solid supports are again retained in place. At this point, each of the 100 sets of solid support carriers will contain 10,000 different compounds. Any necessary reactions will be performed where the reactants used on each compound are the same. The third set of 100 building blocks will then be introduced, one per reaction vessel. This library will then contain 1,000,000 different compounds.

In an alternative embodiment of the present invention illustrated in FIG. 8A, the method described can be extended to a three-dimensional version of a support carrier 11a and solid supports 22. In this embodiment, solid supports 22 in the form of beads or spheres, for example, may be positioned between rods 13 extending upward from the base 15 of support carrier 1a. In FIG. 8B, a second support carrier 1b having extending rods 13 may then be inserted orthogonally in relation to the first support carrier 11a (indicated by the arrow) so as to secure the solid supports 22 in place therein. A barrier or force, as described with reference to the other embodiments, may be used in the three-dimensional embodiment to retain and immobilize the solid supports and prevent the solid supports from moving off a single solid support carrier. Such a barrier could include, but is not limited to, an object positioned at the end of the rods and which blocks the path of the beads or spheres from being removed or falling off of the solid support carrier. The barrier may also consist of a force applied to the beads or spheres to retain the beads or spheres on the rods and may include, but is not limited to a magnet or gravity. When a barrier or force is used to retain the solid supports on a single support carrier during reactions or at other times in which movement of the solid supports is not desired, it would be possible to redistribute the solid supports in two dimensions without the need for a second support carrier. When it is necessary or desired to redistribute the solid supports in the third dimension, the second solid support carrier 11b can be inserted as indicated in FIG. 8B, followed by the removal of the first support carrier.

As with the two-dimensional solid support carrier embodiment described previously, the redistribution of solid supports in the three-dimensional embodiment, from a first solid support carrier to a second, receiving solid support carrier can also be done in parallel simultaneously. In this manner, 100,000,000 solid supports in 100 solid support carriers, each containing 100×100×100 array of one million solid supports, could be distributed in only 100 movements of the solid supports. For example, a barrier could be positioned at the end of the rods of each solid support carrier such that the solid supports form all but one of the rows are prevented from moving off each solid support carrier. Then, the one row which can move off the carrier and which comprises 10,000 solid supports (100 across by 100 deep), is moved in a controlled manner onto each second, receiving solid support carrier in parallel simultaneously. In this manner, 1,000,000 solid supports are transferred simultaneously and only 100 total movements of solid supports are required to redistribute all of the solid supports.

Manipulation of the solid support carriers in the various embodiments of the present invention throughout the chemical syntheses described, may be via manual manipulation or via robotic machinery programmed by computers. The invention is not, however, limited with regard to the mode or manner in which the solid support carriers are manipulated.

An alternative embodiment of the solid support carrier 10 is illustrated in FIG. 9, in which solid supports 22 are held in a two-dimensional array, and in which the solid support carrier 10 has a configuration wherein the solid supports are retained in an array having a corrugated surface having grooves and ridges.

In the methods in accordance with the present invention, the number of times that solid supports are rearrayed on the solid support carriers would typically range between 1 and 7, but could range much higher, especially for the synthesis of oligomers, such as polypeptides or polynucleotides, where the typical range could be as high as 500.

It should also be understood that in the methods as described in the various embodiments herein above, it is not essential that all the solid supports on a solid support carrier are reacted and/or rearranged during the synthesis. Depending upon the protocol, all or only some solid supports may react and/or undergo rearrangement or shuffling during synthesis.

From the foregoing detailed description of the specific embodiments of the invention, it should be apparent that what has been described is a unique apparatus and method by which solid supports may be retained in place in an array within an reaction vessel but which allows easy lateral manipulation of the solid supports in a defined manner to new arrays for subsequent steps in the synthesis of combinatorial chemical libraries of compounds. Although particular embodiments have been disclosed herein in detail, this has been done by way of example for purposes of illustration only, and is not intended to be limiting with respect to the scope of the appended claims that follow, and it will be understood that various omissions, substitutions and changes in the form and details of the disclosed invention maybe made by those skilled in the art without departing from the spirit of the invention. In particular, it is contemplated by the inventor that various substitutions, alterations, and modifications may be made to the invention without departing from the spirit and scope of the invention as defined by the claims.

It is also to be understood that the drawings are not necessarily drawn to scale, but that they are merely conceptual in nature.

What is claimed is:

1. An apparatus for facilitating the spatial arrangement, tracking and identification of solid supports during the formation of a combinatorial chemistry library, comprising:
a solid support carrier for supporting at least one solid support dispersed in a multi-dimensional array thereon, wherein the solid supports can move within said solid support carrier in a controlled manner defined by the shape of said solid support carrier, and wherein the solid supports can move off of said solid support carrier onto other said solid support carriers in a controlled manner defined by the shape of said solid support carriers.

2. The apparatus as recited in claim 1, wherein said solid supports are retained on said solid support carrier in a two-dimensional array.

3. The apparatus as recited in claim 1, wherein said solid supports are retained on said solid support carrier in a three-dimensional array.

4. The apparatus as recited in claim 1, wherein the solid support carrier has a first end and a plurality of elongated tines extending from said first end, said elongated tines defining a plurality of channels formed between said elongated tines within which said solid supports are slidably retained.

5. The apparatus as recited in claim 1, wherein the solid support carrier comprises a plurality of grooves and ridges in which said solid supports are retained.

6. The apparatus as recited in claim 1, wherein a plurality of said solid supports are secured and retained in a spatial arrangement defined by said solid support carrier.

7. The apparatus as recited in claim 1, wherein said solid support carrier is aligned and interfaced with a second solid support carrier such that a plurality of said solid supports are secured and retained in a spatial arrangement defined by said solid support carriers.

8. The apparatus as recited in claim 4, wherein said solid support carrier is aligned and interfaced with a second solid support carrier such that the tines of said second solid support carrier are oriented substantially perpendicular to the tines of said first solid support carrier.

9. The apparatus as recited in claim 4, wherein said solid support carrier is aligned and interfaced with a second solid support carrier such that the tines of said second solid support carrier are oriented approximately 90° to the tines of said first solid support carrier.

10. The apparatus as recited in claim 1, wherein a barrier is positioned on said solid support carrier to prevent the movement of said solid supports off of said solid support carrier.

11. The apparatus as recited in claim 1, wherein said solid supports on said solid support carrier are immersed in a reaction vessel.

12. The apparatus as recited in claim 1, wherein said solid support carrier and said solid supports on said solid support carrier are immersed in a reaction vessel.

13. The apparatus as recited in claim 1, wherein said support carrier may be manipulated manually.

14. The apparatus as recited in claim 1, wherein said solid support carrier may be manipulated by robotic machinery.

15. The apparatus as recited in claim 1, wherein said solid supports are selected from the group consisting of various shapes of polymers, including polystyrene, grafted co-polymers of combinations and composites of polystyrene (optionally cross-linked with divinylbenzene), polypropylene, polyethylene, polyacrylamide and/or polyethylene glycol or substituted variants, polycarbonate, polytetrafluorethylene, Kieselguhr-polyacrylamide non-covalent complex, nylon, glass, silicon, rubber, dextran, chitin, pumice, sand, agarose, polysaccharides, dendrimers, small beads, pellets, disks, capillaries, hollow fibers, needles, solid fibers, cellulose beads, poreglass beads, silica gels, polystyrene beads cross-linked with divinylbenzene and optionally grafted with polyethylene glycol, grafted co-poly beads, poly-acrylamide beads, latex beads, dimethylacrylamide beads optionally cross-linked with N,N′-bisacryloyl ethylene diamine, polydimethylacrylamide beads crosslinked with polystyrene, silica gels, latex beads, glass particles coated with a hydrophobic polymer, or combinations of one or more of the above or other materials used as supports for solid phase synthesis.

16. The apparatus as recited in claim 1, wherein said solid supports may comprise one or more vials.

17. The apparatus as recited in claim 1, wherein each solid support positioned on said solid support carrier is associated with a single reaction vessel.

18. The apparatus as recited in claim 1, wherein a plurality of the solid supports positioned on said solid support carrier is associated with a single reaction vessel.

19. The apparatus as recited in claim 1, wherein a plurality of solid support carriers having solid supports positioned thereon are associated with a single reaction vessel.

20. The apparatus as recited in claim 1, wherein all solid supports positioned on a single solid support carrier is associated with a single reaction vessel.

21. The apparatus as recited in claim 1, comprising a plurality of solid support carriers, each of said solid support carriers capable of supporting at least one solid support dispersed in a multi-dimensional array thereon, wherein the solid supports can move upon said solid support carrier in a controlled manner defined by the shape of said solid support carrier, and wherein the at least one solid support can move off of said solid support carrier onto other said solid support carriers in a controlled manner defined by the shape of said solid support carriers.

22. A method for producing a combinatorial library of compounds, which comprises the steps of:
a. positioning one or more solid supports upon a first series of solid support carriers;
b. reacting a subset of monomers, compounds or building blocks with said solid supports on said solid support carriers, in a series of reaction vessels to define a first series of arrays of said solid supports;
c. redistributing the first series of arrays of solid supports into a second series of arrays of solid supports by sliding said solid supports from said first solid support carrier onto said second solid support carrier;
d. repeating steps (a)-(c) between 1-500 times; and
e. identifying every individual compound in the library by analyzing the position of each solid support in each array at each step in the library synthesis.

23. The method as recited in claim 22, wherein said solid support carriers comprise a first end and a plurality of tines extending from said first end and having a plurality of channels defined between said tines within which said solid supports are slidably retained.

24. The method as recited in claim 22, wherein the solid support carriers have a plurality of grooves and ridges on which said solid supports are retained.

25. The method as recited in claim 22, wherein said redistributing step permits the manipulation of said solid supports laterally within an x-y plane in said channels and along said tines.

26. The method as recited in claim 22, wherein said redistributing step permits the manipulation of said solid supports laterally within an x-y-z space in said channels and along said tines.

27. The method as recited in claim 22, wherein the identity of each solid support in the identifying step can be determined by its position within the array defined by the channels in said support carriers.

28. The method as recited in claim 22, wherein all the solid supports undergo chemical reactions during said reacting step.

29. The method as recited in claim 22, wherein only a portion of the solid supports undergo chemical reactions during said reacting step.

30. The method as recited in claim 22, wherein said solid support carriers may be manipulated manually.

31. The method as recited in claim 22, wherein said solid support carriers may be manipulated via robotic machinery.

32. An apparatus for facilitating the spatial arrangement, tracking and identification of solid supports during the formation of a combinatorial chemistry library, which comprises:
a first solid support carrier having a first end and a plurality of elongated tines extending from said first end, said elongated tines defining a plurality of channels formed between said tines, wherein said of solid supports are slidably positioned in said channels, and
a second solid support carrier having a first end and a plurality of elongated tines extending from said first end, said tines defining a plurality of channels formed between each of said tines, such that said solid supports may be retained and immobilized when said second solid support carrier is aligned and interfaced with said first solid support carrier.

33. The apparatus as recited in claim 32, wherein said first solid support carrier and said second solid support carrier are aligned and interfaced such that the tines of said second solid support carrier are oriented substantially perpendicular to the tines of said first solid support carrier.

34. The apparatus as recited in claim 32, wherein said first solid support carrier and said second solid support carrier are interfaced such that the tines of said second solid support carrier are oriented approximately 90° to the tines of said first solid support carrier.

35. The apparatus as recited in claim 32, wherein said solid supports on said first and second solid support carriers are immersed in a reaction vessel.

36. The apparatus as recited in claim 32, wherein said first and second solid support carriers and said solid supports on said first and second solid support carriers are immersed in a reaction vessel.

37. The apparatus as recited in claim 32, wherein said first and second support carriers may be manipulated manually.

38. The apparatus as recited in claim 32, wherein said first and second solid support carriers may be manipulated by robotic machinery.

39. The apparatus as recited in claim 32, wherein said solid supports are selected from the group consisting of various shapes of polymers, including polystyrene, grafted co-polymers of combinations of polystyrene (optionally crosslinked with divinylbenzene), polypropylene and/or polyethylene glycol or substituted variants, small beads, pellets, disks, capillaries, hollow fibers, needles, solid fibers, cellulose beads, pore-glass beads, silica gels, polystyrene beads cross-linked with divinylbenzene and optionally grafted with polyethylene glycol, grafted co-poly beads, poly-acrylamide beads, latex beads, dimethylacrylamide beads optionally cross-linked with N,N′-bis-acryloyl ethylene diamine, polydimethylacrylamide beads crosslinked with polystyrene, glass particles coated with a hydrophobic polymer, or combinations of one or more of the above.

40. The apparatus as recited in claim 32, wherein solid supports may comprise one or more vials.

41. The apparatus as recited in claim 32, comprising a plurality of first and second solid support carriers, each of said first and second solid support carriers having a first end and a plurality of elongated tines extending from said first end, said elongated tines defining a plurality of channels formed between said tines, within which said of solid supports are slidably retained.

42. An apparatus for facilitating the spatial arrangement, tracking and identification of solid supports during the formation of a combinatorial chemistry library, comprising a solid support carrier for supporting one or more solid supports dispersed in a multi-dimensional array thereon, said solid supports having the ability to move within said solid support carrier in a controlled manner defined by the shape of said solid support carrier, and wherein a barrier is provided to prevent the movement of said solid supports on said solid support carrier, such that removal of said barrier allows the solid supports to move off of said solid support carrier onto other said solid support carriers in a controlled manner defined by the shape of said solid support carrier.

43. The apparatus as recited in claim 42, wherein a plurality of solid supports are secured and retained in a spatial arrangement defined by said solid support carrier.

44. The apparatus as recited in claim 42, wherein said barrier may comprise a second solid support carrier.

45. The apparatus as recited in claim 42, wherein said solid support carrier may be manipulated manually.

46. The apparatus as recited in claim 42, wherein said solid support carrier may be manipulated by robotic machinery.

47. The apparatus as recited in claim 42, wherein said solid supports are retained on said solid support carrier in a two-dimensional X-Y array.

48. The apparatus as recited in claim 42, wherein said solid supports are retained on said solid support carrier in a three-dimensional X-Y-Z array.

49. The apparatus as recited in claim 42, comprising a plurality of solid support carriers, each capable of supporting one or more solid supports dispersed in a multidimensional array thereon, said solid supports having the ability to move within said solid support carriers in a controlled manner defined by the shape of said solid support carriers, and wherein a barrier is provided to prevent the movement of said solid supports on said solid support carriers, such that removal of said barrier allows the solid supports to move off of said solid support carriers onto other said solid support carriers in a controlled manner defined by the shape of said solid support carrier.

50. A method for producing a combinatorial library of compounds, which comprises the steps of:
a. positioning one or more solid supports upon a first series of solid support carriers, each solid support carrier further comprising a barrier positioned to retain said solid supports on said solid support carriers;
b. reacting a subset of monomers, compounds or building blocks with said solid supports on said solid support carriers, in a series of reaction vessels such that there is one array per reaction vessel;
c. removing said barrier from said solid support carrier and redistributing the first series of arrays of solid supports into a second series of arrays of solid supports by sliding said solid supports from said first solid support carrier onto a second solid support carrier;
d. repositioning said solid support barrier on said solid support carrier to retain said solid supports thereon;
e. redistributing the first series of arrays of solid supports into a second series of arrays of solid supports by sliding said solid supports from the second solid support carrier onto a next solid support carrier;
f. repeating steps (a)-(d) between 1 and 500 times; and
g. identifying every individual compound in the library by analyzing the position of each solid support in each array at each step in the library synthesis.

51. The method as recited in claim 50, wherein said solid support carriers have a first end and a plurality of elongated tines extending from said first end, said elongated tines defining a plurality of channels formed between said elongated tines within which said solid supports are slidably retained.

52. The method as recited in claim 51, wherein the identity of each solid support in the identifying step can be determined by its position with the array defined by the channels in said solid support carriers.

53. The method as recited in claim 51, wherein all solid supports undergo chemical reactions during said reacting step.

54. The method as recited in claim 50, wherein only a portion of said solid supports undergo chemical reactions during said reacting step.

55. The method as recited in claim 50, wherein said solid support carriers may be manipulated manually.

56. The method as recited in claim 50, wherein said solid support carriers may be manipulated by robotic machinery.
